(19) United States
(12) Patent Application Publication
(10) Pub. No.: US 2022/0000893 A1
(43) Pub. Date: Jan. 6, 2022

(54) METHOD FOR TREATING T-HELPER TYPE 2 MEDIATED DISEASE

(71) Applicants: |NSERM (INSTITUT NATIONAL DE LA SANTÉ ET DE LA RECHERCHE MÉDICALE), Paris (FR); UNIVERSITÉ PAUL SABATIER TOULOUSE III, Toulouse (JP); CENTRE NATIONAL DE LA RECHERCHE SCIENTIFIQUE (CNRS), Paris (FR)

(72) Inventors: Olivier JOFFRE, Toulouse Cedex 3 (FR); Bénédicte BINET, Toulouse Cedex 3 (FR); Véronique ADOUE, Toulouse Cedex 3 (FR); Agathe MALBEC, Toulouse Cedex 3 (FR); Karl LAVIOLETTE, Toulouse Cedex 3 (FR)

(21) Appl. No.: 17/289,413

(22) PCT Filed: Oct. 30, 2019
(86) PCT No.: PCT/EP2019/079603

§ 371 Date: Apr. 28, 2021

(30) Foreign Application Priority Data
Oct. 31, 2018 (EP) 18306432.8

(51) Int. Cl. A61K 31/704; A61K 31/437; A61K 31/337; A61K 39/39; A61P 37/08

(52) U.S. Cl. CPC A61K 31/704; A61K 31/437; A61K 2039/55572; A61K 39/39; A61P 37/08; A61K 31/337

(57) ABSTRACT

The present invention relates to the treatment of T-helper type 2 (Th2)-mediated disease. Here, the inventors set out to investigate at the genome level the effects of SETDB1-dependent H3K9me3 deposition on CD4 T cell activation, differentiation and commitment. By using conditional Setdb1−/− mice, they show that SETDB1 restricts Th1 cell priming and ensures Th2 cell integrity. Unlike their wild-type counterparts, SETDB1-deficient Th2 cells readily express the entire Th1 gene network when exposed to the Th1-instructing cytokine IL-12. More, SETDB1 methylates H3K9 at a subset of ERVs that flank and repress Th1 enhancers or behave themselves as cis-regulatory elements of a large network of Th1 genes, including Ifng, Stat4, Runx3 and Tbx21. Therefore, H3K9me3 deposition by SETDB1 locks the Th1 gene expression program and thus ensures T cell lineage integrity by repressing a repertoire of ERVs that have been co-opted to behave as Th1 lineage-specific cis-regulatory modules. Thus, the invention relates to a SETDB1 inhibitor for use in a method for increasing the Th1/Th2 ratio of an immune response in a subject in need thereof.

### FIELD OF THE INVENTION

The present invention relates to a SETDB1 inhibitor for use in a method for increasing the Th1/Th2 ratio of an immune response in a subject in need thereof.

### BACKGROUND OF THE INVENTION:

T lymphocytes protect vertebrates against a wide variety of endogenous and exogenous dangers, comprising tumors, viruses, bacteria and parasites. Their efficacy comes at least in part from their ability to adapt their phenotype and function to the threat detected by the cells of the innate immune system. Depending on the nature and strength of the signals delivered by these cells and the surrounding tissues, T lymphocytes mobilize different networks of transcription factors, resulting in induction of distinct developmental programs that coordinate the acquisition of lineage-specific and danger-adapted phenotypes and functions (O'Shea and Paul, 2010; Wilson et al., 2009). This plasticity is best illustrated by naïve CD4 T cells, which, upon activation, are able to differentiate into a large and probably still underestimated number of distinct effector populations.

The transcription factors mobilized in response to environmental signals orchestrate a massive remodeling of the epigenetic landscape of T cells (Kanno et al., 2012; Wilson et al., 2009). These dynamic changes in chromatin composition and compaction are necessary to set up and stabilize gene expression programs and to allow their faithful transmission to the progeny. Indeed, interfering with the post-translational modifications of histones or with DNA methylation critically affects the differentiation and stability of effector and memory T cells (Allan et al., 2012; Tumes et al., 2013; Wilson et al., 2009; Xiao et al., 2016; Young et al., 1994). In CD4 T lymphocytes, epigenetic remodeling is largely coordinated by signal-transducer-and-activator-of-transcription (STAT) proteins and by the master regulators specific to each lineage, such as T-bet and GATA-3 for Th1 and Th2 cells, respectively (Kanno et al., 2012; O'Shea et al., 2011). These transcriptional regulators fine-tune the balance between T cell determination and plasticity by directing the deposition of permissive epigenetic marks at lineage-specific cis-regulatory elements, and by targeting repressive epigenetic pathways to the loci associated with alternative fates (Kanno et al., 2012; O'Shea et al., 2011; Vahedi et al., 2012; Wilson et al., 2009).

Among the various post-translational modifications found on the N-terminal tail of core histones, lysine methylation has emerged as a key modification that controls genome functions (Mozzetta et al., 2015). In contrast to histone acetylation, which is almost systematically associated with transcriptional activation, the consequences of histone methylation depend on the residue that is targeted, on its level of methylation, and on the position of the methylated histone in the genome. Methylation of histone H3 on Lys 4 (H3K4), Lys 36 (H3K36) or Lys 79 (H3K79), for example, is associated with active transcription, whereas trimethylated Lys 27 (H3K27me3) accumulates at loci that are either repressed or poised for transcription (Mozzetta et al., 2015). The consequences of H3K9me3 on nuclear function are more complex. This modification was first implicated in the scaffolding, silencing, and function of constitutive heterochromatin at pericentromeric and telomeric regions (García-Cao et al., 2003; Lachner et al., 2001; Peters et al., 2001). More recently, H3K9me3 has been shown to restrict developmental potency of the embryo (Becker et al., 2016). Its deposition at promoters of genes encoding developmental regulators has also been described as necessary to repress these loci and maintain embryonic stem cells pluripotency (Becker et al., 2016; Bilodeau et al., 2009). In adult cells, H3K9me3-dependent repression of gene expression in euchromatin and facultative heterochromatin is also important to define and maintain cell identity (Allan et al., 2012; Becker et al., 2016; Liu et al., 2015). However, the repertoires of loci and genomic elements that are targeted as well as the molecular mechanisms at work remain poorly characterized. H3K9me3 finally accumulates on the body of active genes where it may potentially affect transcription elongation and alternative splicing (Saint-André et al., 2011; Vakoc et al., 2005). H3K9me3 is thus a versatile chromatin mark that has multiple and sometimes opposing functions depending on the type and differentiation status of the cell and on its location at functionally distinct elements of the genome.

Several lysine methyltransferases can trimethylate H3K9. They include SUV39H1, SUV39H2 and SETDB1, which all belong to the SUV39H family (Mozzetta et al., 2015; Peters et al., 2001; Schultz et al., 2002). Whereas SUV39H1 and SUV39H2 were first identified as key components of constitutive heterochromatin (García-Cao et al., 2003; Peters et al., 2001; 2002), SETDB1 was initially found to be involved in the dynamic repression of gene transcription at euchromatin and facultative heterochromatin (Schultz et al., 2002). SUV39H1 has since been shown to repress euchromatic gene expression through H3K9me3 deposition at promoters (Allan et al., 2012; Liu et al., 2015), and SETDB1 has been implicated in constitutive heterochromatin organization (Loyola et al., 2009). In fact, the maintenance of H3K9me3 at pericentromeric heterochromatin during DNA replication depends on a stepwise process involving H3K9 mono- and tri-methylation by SETDB1 and SUV39H1, respectively (Loyola et al., 2009; 2006). In embryonic stem cells, the two H3K9-specific methyltransferases also collaborate to repress endogenous retroviruses (ERVs). SETDB1 initiates the deposition of H3K9me3 at this class of transposable elements and these sites are subsequently extended into broad domains by SUV39H1 (Bulut-Karslioglu et al., 2014). Interestingly, very recent data provided evidence that ERVs have been co-opted as cis-regulatory elements to shape and control gene networks in various cell types (Chuong et al., 2017). SETDB1 and SUV39H1 may therefore also control cell integrity through deposition of H3K9me3 at these genomic elements. In conclusion, the genomic locations and functions of the different enzymes that trimethylate H3K9 are not as mutually exclusive as initially thought; while SUV39H1/2 and SETDB1 have non-redundant functions, they can also collaborate to fine-tune cell identity and fate in response to environmental signals.

The studies mentioned above highlight the general complexity and importance of H3K9me3-dependent epigenetic regulatory pathways in genome function. In T cells, more specifically, SUV39H1 has been implicated in Th2 cell stability by depositing H3K9me3 at the Ifng promoter (Allan et al., 2012). However, as IFN-γ is not sufficient to reprogram fully committed Th2 cells to adopt a Th1 phenotype (Hegazy et al., 2010), the deregulation of the Ifng locus observed in Suv39h1−/− cells cannot, only by itself, explains a loss of Th2 cell integrity. Other critical Th1 cell lineage-specific loci might therefore be controlled by H3K9me3-dependent repressive mechanisms. Interestingly, while a clear H3K9me3 signal is detected at the Tbx21 gene in Th2 cells, SUV39H1 has no impact in the deposition of the repressive mark at this locus which encodes T-bet, the master regulator of the Th1 lineage (Allan et al., 2012). Together with the fact that H3K9me3 disappearance at euchromatin and facultative heterochromatin is very limited in SUV39H1-deficient cells (Peters et al., 2002), these observations suggest that other H3K9me3-dependent epigenetic pathways play a critical role in controlling Th2 cell stability.

### SUMMARY OF THE INVENTION

Here, the inventors set out to investigate at the genome level the effects of SETDB1-dependent H3K9me3 deposition on CD4 T cell activation, differentiation and commitment. To date, SETDB1 had only been implicated in OX40-dependent repression of the Il17a locus in Th17 cells (Xiao et al., 2016). By using conditional Setdb1−/− mice, they show that SETDB1 restricts Th1 cell priming and ensures Th2 cell integrity. Unlike their wild-type counterparts, SETDB1-deficient Th2 cells readily express the entire Th1 gene network when exposed to the Th1-instructing cytokine IL-12. Interestingly, SETDB1 does not repress Th1-related loci by depositing H3K9me3 at gene promoters, as previously reported for SUV39H1 at the Ifng locus (Allan et al., 2012). Instead, it methylates H3K9 at a subset of ERVs that flank and repress Th1 enhancers or behave themselves as cis-regulatory elements of a large network of Th1 genes, including Ifng, Stat4, Runx3 and Tbx21. Therefore, H3K9me3 deposition by SETDB1 locks the Th1 gene expression program and thus ensures T cell lineage integrity by repressing a repertoire of ERVs that have been co-opted to behave as Th1 lineage-specific cis-regulatory modules.

Thus, the present invention relates to a SETDB1 inhibitor for use in a method for increasing the Th1/Th2 ratio of an immune response in a subject in need thereof. Particularly, the invention is described by its claims.

### DETAILED DESCRIPTION OF THE INVENTION

A SETDB1 inhibitor for use in a method for increasing the Th1/Th2 ratio of an immune response in a subject in need thereof.

The inventors showed, by using a conditional Setdb1−/− mice that the Th1 response is increasing (showed by the increasing of Th1 markers like GM-CSF and IFNγ). Inhibition could be thus useful for boosting the Th1 response in disease in need thereof like cancer and infectious diseases, during a vaccine protocol or for the treatment of T-helper type 2 (Th2)-mediated disease like allergic disorder or, asthma, in particular allergic asthma.

By “increasing the Th1/Th2 ratio of an immune response” is included the meaning that the ratio of mouse Immunoglobulin G subclass 2a (IgG2a) concentrations to mouse IgG1 concentrations for a chosen antigen is increased. These concentrations correlate with Th1 and with Th2 profiles (Mosmann T. R. and Coffman R. L. 1989). The concentration of mouse IgG2a may increase and/or the concentration of mouse IgG1 may decrease. In human, the same correlations apply to IgG1/3 and to IgG4 as they are associated with Th1 and Th2 immune responses, respectively. IFNγ or TNF production, IL-4, IL-5, IL-13, Amphiregulin and/or IgE levels, or levels of other cytokines, may also be used in assessing the Th1/Th2 ratio. For example, to examine antigen-specific T cell responses, an ex vivo assay that measures spleen, lymph nodes, or tissue-infiltrating T cell production of Th1- or Th2-related cytokines upon antigen-specific restimulation may be used. The Th1/Th2 cytokine profiles might be measured either by analysis of the cell culture supernatant or by intracellular staining and flow cytometry. IFNγ production is indicative of a Th1 response, whilst IL-4, IL-5 or IL-13 production is indicative of a Th2 response. Frequency, absolute numbers or activation status of eosinophils, innate lymphoid cells, macrophages or any other immune cells may also be used in assessing T helper cell polarization. The expression of chemokine receptors by CD4+ T cell, including CCR5 and CXCR3 for human Th1 cells, and CCR3, CCR4 and CCR8 for human Th2 cells, might also be used in determining the Th1/Th2 balance.

Thus the invention also relates to a SETDB1 inhibitor for boosting a Th1 response of an immune response. In other word, the invention relates to a SETDB1 inhibitor for use in the treatment of a disease wherein the Th1 could be beneficial. Such disease can be cancer or infectious disease.

Thus, the invention also relates to a SETDB1 inhibitor for use in the treatment of cancer or infectious diseases by boosting the Th1 response.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyo sarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Examples of infection diseases include, but are not limited to, viral, bacterial, parasitic or fungal infections such as chronic hepatitis, lung infections, lower respiratory tract infections, bronchitis, influenza, pneumoniae and sexually transmitted diseases.

Examples of viral infections include, but are not limited to, hepatitis (HAV, HBV, HCV), herpes simplex (HSV), herpes zoster, HPV, influenza (Flu), AIDS and AIDS related complex, chickenpox (varicella), common cold, cytomegalovirus (CMV) infection, smallpox (variola), Colorado tick fever, dengue fever, ebola hemorrhagic fever, foot and mouth disease, lassa fever, measles, marburg hemorrhagic fever, infectious mononucleosis, mumps, norovirus, poliomyelitis, progressive multifocal leukoencephalopathy (PML), rabies, rubella, SARS, viral encephalitis, viral gastroenteritis, viral meningitis, viral pneumonia, West Nile disease and yellow fever.

Examples of bacterial infections include, but are not limited to, pneumonia, bacterial meningitis, cholera, diphtheria, tuberculosis, anthrax, botulism, brucellosis, campylobacteriosis, typhus, gonorrhea, listeriosis, lyme disease, rheumatic fever, pertussis (Whooping Cough), plague, salmonellosis, scarlet fever, shigellosis, syphilis, tetanus, trachoma, tularemia, typhoid fever, and urinary tract infections.

Examples of parasitic infections include, but are not limited to, malaria, leishmaniasis, trypanosomiasis, chagas disease, cryptosporidiosis, fascioliasis, filariasis, amebic infections, giardiasis, pinworm infection, schistosomiasis, taeniasis, toxoplasmosis, trichinellosis, and trypanosomiasis. Examples of fungal infections include, but are not limited to, candidiasis, aspergillosis, coccidioidomycosis, cryptococcosis, histoplasmosis and tinea pedis.

The invention also relates to a SETDB1 inhibitor for use in the treatment of a T-helper type 2 (Th2)-mediated disease in a subject in need thereof.

As used herein, the term “SETDB1” has its general meaning in the art and refers to the histone H3, lysine 9-specific methyltransferase “SET Domain Bifurcated 1” (Schultz et al., 2002), also known as KMT1E or ERG-associated protein with SET domain (ESET).

The terms “SETDB1 inhibitor” denotes molecules or compound which can inhibit the activity of the proteins (e.g. inhibit the transferase activity of the proteins) or a molecule or compound which destabilizes the proteins. In particular, an inhibitor of SETDB1 can inhibit the methylation activity of the enzyme on particular histone. Particularly, an inhibitor of SETDB1 can inhibit the methylation of Lys-9 of histone H3 by the methyltransferase SETDB1.

The term “SETDB1 inhibitor” also denotes inhibitors of the expression of the gene coding for the proteins. In order to test the functionality of a putative SETDB1 inhibitor a test is necessary. For that purpose, to identify SETDB1 inhibitors, H3K9me3 levels might be assessed in treated and controlled cell lines by intracellular staining and flow cytometry. Alternatively, global H3K9me3 levels might also be quantified by western-blotting or fluorescent microscopy. To test if the putative inhibitor directly inhibits SETDB1, H3-specific histone methyl transferase assay might be performed using purified SETDB1 protein, tritiated S-adenosyl-methionine (S-adenosyl-L-[methyl-^{H}3]methionine) and purified histone octamer, mononucleosomes or oligonucleosomes as histone substrate. If the SETDB1 inhibitor can impact on the expression of the lysine methyltransferase, SETDB1 expression might be determined at the mRNA or protein level using semi-quantitative PCR, flow cytometry, or western-blotting.

As used herein, the term “subject” denotes a mammal, such as a rodent, a feline, a canine, and a primate. Particularly, the subject according to the invention is a human.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “T-helper type 2 (Th2)-mediated disease” means a disease which is characterized by the overproduction of Th2 cytokines like IL4, including those that result from an overproduction or bias in the differentiation of T-cells into the Th2 subtype. Such diseases include, for example, exacerbation of infection with infectious diseases (e.g., Leishmania major, Mycobacterium leprae, Candida albicans, Toxoplasma gondi, respiratory syncytial virus, human immunodeficiency virus, etc.) and allergic disorders, such as anaphylactic hypersensitivity, asthma, allergic rhinitis, atopic dermatitis, vernal conjunctivitis, eczema, urticaria and food allergies, etc. More particularly, Th2-mediated diseases include but are not limited to graft immune diseases (chronic GVHD), autoimmune diseases (especially organ non-specific autoimmune diseases) and type-Th2 allergic diseases. Diseases exemplified typically are ulcerative colitis, systemic lupus erythematosus, myasthenia gravis, systemic progressive scleroderma, rheumatoid arthritis, interstitial cystitis, Hashimoto's diseases, Basedow's diseases, autoimmune hemolytic anemia, idiopathic thrombocytopenic purpura, Goodpasture's syndrome, atrophic gastritis, pernicious anemia, Addison diseases, pemphigus, pemphigoid, lenticular uveitis, sympathetic ophthalmia, primary biliary cirrhosis, active chronic hepatitis, Sjogren's syndrome, multiple myositis, dermatomyositis, polyarteritis nodosa, rheumatic fever, glomerular nephritis (lupus nephritis, IgA nephropathy, and the like), allergic encephalitis, atopic allergic diseases (for example, bronchial asthma, allergic rhinitis, allergic dermatitis, allergic conjunctivitis, pollinosis, urticaria, food allergy and the like), Omenn's syndrome, vernal conjunctivitis and hypereosinophilic syndrome.

As used herein, terms such as “Th1 cell” and/or “Th1 phenotype” and all grammatical variations thereof refer to a differentiated CD4⁺ T helper cell that expresses interferon gamma (IFN-γ).

In a particular embodiment, the SETDB1 inhibitor according to the invention is used for the treatment of an allergic disorder, asthma, in particular allergic asthma.

In a particular embodiment, the SETDB1 inhibitor is used in combination with any immune adjuvant inducting and/or promoting Th1 cell differentiation (e.g. Th1 adjuvant).

Thus, the invention also relates to a i) SETDB1 inhibitor, and ii) a Th1 adjuvant, as a combined preparation for simultaneous, separate or sequential use in the treatment of a T-helper type 2 (Th2)-mediated disease in a subject in need thereof.

As used herein, the Th1 adjuvants are selected in the group consisting in but not limited to IL-12, LPS, Complete Freund's adjuvant, Aluminium salts like Alum, CpG, squalene (see for example Coffman R L et al., Immunity 2010).

In one embodiment, the inhibitors according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In a particular embodiment, the SETDB1 inhibitor is selected from the group consisting of mithramycin (also known as plicamycin), mithramycin analogs, 3-Deazaneplanocin A (also known as DZNep) and paclitaxel (see for example Karanth A R et al 2017).

In one embodiment, the inhibitor according to the invention (inhibitor of SETDB1) is an antibody. Antibodies or directed against SETDB1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against SETDB1 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-SETDB1 single chain antibodies. Compounds useful in practicing the present invention also include anti-SETDB1 antibody fragments including but not limited to F(ab′)2 fragments, which can be generated by pepsin digestion of an intact antibody molecule, and Fab fragments, which can be generated by reducing the disulfide bridges of the F(ab')2 fragments. Alternatively, Fab and/or scFv expression libraries can be constructed to allow rapid identification of fragments having the desired specificity to SETDB1.

Humanized anti-SETDB1 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of SETDB1 is selected.

In a particular embodiment, the anti-SETDB1 antibody according to the invention may be the 5H6A12 or K.137.7 antibodies as send by Thermofisher.

In another embodiment, the antibody according to the invention is a single domain antibody against SETDB1. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamer of SETDB1 is selected.

In one embodiment, the compound according to the invention is a polypeptide.

In a particular embodiment the polypeptide is an antagonist of SETDB1 and is capable to prevent the function of SETDB1. Particularly, the polypeptide can be a mutated SETDB1 protein or a similar protein without the function of SETDB1.

In one embodiment, the polypeptide of the invention may be linked to a “cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 Daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomerular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the SETDB1 inhibitor according to the invention is an inhibitor of SETDB1 gene expression.

Small inhibitory RNAs (siRNAs) can also function as inhibitors of SETDB1 expression for use in the present invention. SETDB1 gene expression can be reduced by contacting a subject or cell with a small double stranded RNA (dsRNA), or a vector or construct causing the production of a small double stranded RNA, such that SETDB1 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of SETDB1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of SETDB1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of SETDB1 gene expression can be prepared by known methods. These include techniques for chemical synthesis such as, e.g., by solid phase phosphoramidite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing SETDB1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Ban viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In another embodiment, the invention relates to a method for increasing the Th1/Th2 ratio of an immune response comprising administering to a subject in need thereof a therapeutically effective amount of a SETDB1 inhibitor.

In another embodiment, the invention relates to a method for treating a T-helper type 2 (Th2)-mediated disease comprising administering to a subject in need thereof a therapeutically effective amount of a SETDB1 inhibitor.

By a “therapeutically effective amount” is meant a sufficient amount of the inhibitor of SETDB1 to treat a T-helper type 2 (Th2)-mediated disease at a reasonable benefit/risk ratio applicable to any medical treatment.

### Therapeutic Composition

Another object of the invention relates a therapeutic composition comprising a SETDB1 inhibitor according to the invention for increasing the Th1/Th2 ratio of an immune response in a subject in need thereof.

Particularly, the invention relates to a therapeutic composition comprising a SETDB1 inhibitor according to the invention for use in the treatment of treating a T-helper type 2 (Th2)-mediated disease in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an inhibitor according to the invention and a further therapeutic active agent, particularly an anti-inflammatory compound. For example, these agents can be nonsteroidal anti-inflammatory drugs like aspirin, ibuprofen, and naproxen, β-agonists, corticoids, anti-histaminics, antileukotrienne, antibodies anti-IgE, anti-IL5 or anti-IL4Ra (see for example Akdis CA, 2012).

The invention will be further illustrated by the following figures and examples. However, these examples and figures should not be interpreted in any way as limiting the scope of the present invention.

### FIGURES:

FIG. 1. Enhanced Th1 priming in the absence of SETDB1.

(A) Average expression (Geometric mean) of T-bet by Setdb1+/+ and Setdb1−/− CD4 T cells after 6 days of culture in Th1 medium containing increasing concentrations of IL-12. (B, C) Percentage of CD4 T cells-producing cytokine (left) and average cytokine production (Geometric mean) per cell (right) after 6 days of culture in Th1 medium containing increasing concentrations of IL-12. (D) Production of cytokines by Setdb1+/+ and Setdb1−/− CD4 T cells following 6 days of culture in Th1-inducing conditions and overnight restimulation with anti-CD3ε and anti-CD28 antibodies. Data are represented as mean±SD of three (D) independent experiments or of three biological replicates from one representative experiment out of three performed (A, B, C). *p<0.05, **p<0.01, ***p<0.001 (unpaired Student's t-test).

FIG. 2. SETDB1 is required for stable Th2 cell commitment.

Setdb1^{+/+} and Setdb1^{-/-} naïve CD4 T cells were cultured for six days in Th2-polarizing conditions, extensively washed in complete medium, and then cultured in Th1-polarizing conditions for two more days. Percentages of cells producing IL-13 and/or IFN-γ as determined by intracellular immunostaining and flow cytometry. Data are representative as mean±SEM of eight independent experiments.

FIG. 3. Non-specific SETDB1 inhibitors alter Th2 cell commitment.

Naïve CD4 T cells were isolated from C57BL/6 mice and were cultured for 6 days in Th2-polarizing conditions in the presence (Mithramycine A, DZNep) or absence (DMSO) of SETDB1 inhibitors. Cells were exposed to 100 nM Mithramycine A and to 1 nM DZNep for the first 4 and 24 hours, respectively. At the end of the culture, cytokine production was assessed by flow cytometry following T cell restimulation with PMA/Ionomycine in the presence of Monensin and intracellular staining. (A, C) Representative flow cytometry profiles and (B, D) frequency of IFNg-producing cells in each experiment (n=2) are shown.

### EXAMPLE

### Material & Methods

### Mice

Suv39h1-deficient mice were kindly provided by T. Jenuwein (Peters et al., 2001). The Setdb1 mutant mouse strain (common strain name EPD0028_1_B07; international strain designation B6Dnk;B6N-Setdb1tm1a(EUCOMM)Wtsi) was established as part of the International Mouse Phenotyping Consortium (EMMA ID: EM:04052) at the German Research Center for Environmental Health (Helmholtz Zentrum, Muenchen). The targeting vector was composed of the promoterless L1L2_gt1 cassette inserted in the L3L4_pZero_kan plasmid backbone. The construct was microinjected into C57BL/6 ES cells (JM8.N4 parental cell line) and the L1L2_gt1 cassette was inserted at position 95350414 of chromosome 3, upstream of Setdb1 exon 4. The cassette was composed of a lacZ-neomycin sequence flanked by Flp Recombinase Target (FRT) sites and followed by a loxP sequence. An additional loxP site was inserted downstream of Setdb1 exon 4 at position 95349598. Additional information on the Setdb1 mutant mouse strain can be found at https://www.infrafrontier.eu/search?keyword=EM:04052. Mice with a conditional ready Setdb1 allele (Setdb1f1) were generated by intercrossing Setdb1 mutant mice with mice expressing the Flipper recombinase under the control of the ubiquitous Rosa26 promoter. Conditional knockout mice (Setdb1−/−) were obtained by intercrossing Setdb1f1/f1 and CD4-CRE mutant mice. All the mice were bred and housed at the Regional Centre of Functional Exploration and Experimental Resources (CREFRE, UMS006/INSERM). Sex-matched 6- to 12-week-old mice were used and compared in all experiments. All experiments involving animals were conducted according to animal study protocols approved by the local ethics committee (#16-U1043-JVM-496 and 16-U1043-JVM-20).

### Naïve CD4+ T Cell Isolation

Spleen and lymph nodes (mesenteric, inguinal, axillary, brachial and cervical) were collected and digested with Liberase TM and DNAse I (Sigma). Single-cell suspensions were then pooled and depleted of erythrocytes by osmotic shock (Red Blood Cell Lysis buffer, Sigma). CD4 T cells were enriched by negative selection by using antibodies specific for CD16/32 (2.4G2), I-A/I-E (M5/114.15.2), CD8a (H59) and B220 (RA3-6B2), and Dynabeads sheep anti-rat IgG (Thermo Fischer Scientific). Naïve CD4 T cells, defined as CD4+CD25-CD62LhighCD44low, were labeled with fluorochrome-conjugated monoclonal antibodies specific for CD4 (GK1.5, BD Biosciences), CD25 (PC61, BD Biosciences), CD62L (MEL14, Thermo Fischer Scientific) and CD44 (IM7, BD Biosciences), and purified from the enriched fraction of CD4 T cells by fluorescence-activated cell sorting (FACS Aria, BD Biosciences).

### T helper Cell cultures

Naïve CD4 T cells were cultured for three days in 96-well flat bottom plates coated with 10 μg/mL anti-CDR antibody (145-2C11, InVivoMab™, BioXcell) in RPMI 1640 Glutamax™ supplemented with 1 mM sodium pyruvate, non-essential amino acids, 10 mM HEPES, 100 units/mL penicillin, 100μg/mL streptomycin, 50 mM 2β-mercaptoethanol, 10% fetal calf serum (all from Thermo Fischer Scientific) and 1 μg/mL anti-CD28 antibody (37.51, InVivoMab™, BioXcell). Unless stated otherwise, Th1 medium also contained 10 ng/mL recombinant mouse IL-12 (R&D Systems) and 10 μg/mL anti-IL-4 neutralizing antibody (11B11, InVivoMab™, BioXcell). Th2 medium contained 50 ng/mL recombinant mouse IL-4 (R&D systems) and 10 μg/mL anti-IFN-γ neutralizing antibody (XMG1.2, InVivoMab™, BioXcell). At day 3, the cells were re-plated in the same conditioning medium but without the anti-CD3ε and anti-CD28 antibodies and with 30 IU/mL recombinant IL-2 (Proleukin). To test for Th2 cell lineage commitment, cells were harvested at day 6, extensively washed in complete medium, and re-plated in Th1-polarizing conditions as indicated above. To assess the role of the IFN-γ pathway in Th2 cell plasticity, Th1 medium was supplemented with 10 μg/mL anti-IFN-γ. In co-culture experiments, Setdb1+/+ and Setdb1−/− Th2 cells were differentiated separately, mixed at a 1:3 ratio, and then plated in Th1 culture conditions.

### T Cell Proliferation and Differentiation Analysis by Flow Cytometry

To analyze intracellular transcription factor expression upon T helper cell differentiation, cells were collected at the requires time points, stained with the fixable viability dye eFluor 506 (Thermo Fischer Scientific), and labeled with fluorochrome-conjugated antibodies specific for T-bet (ebio4B10, Thermo Fischer Scientific) and GATA-3 (TWAJ, Thermo Fischer Scientific) by means of the Transcription Factor Staining Buffer Set (Thermo Fischer Scientific). For intracellular cytokine staining, cells were first stimulated at 37° C. with 20 ng/mL phorbol 12-myristate 13-acetate (Millipore) and 1 μg/mL ionomycin (Millipore) for 5 hours in the presence of GolgiStop™ (BD Biosciences). Cells were then labelled with the fixable viability dye eFluor 506 and stained with fluorochrome-coupled antibodies specific for IL-13 (ebiol3A, Thermo Fischer Scientific), IFN-γ (XMG1.2, Thermo Fischer Scientific), GM-CSF (MP1-22E9, BD Biosciences) or TNF (MP6-XT22, Thermo Fischer Scientific) by using the Cytofix/Cytoperm™ Fixation/Permeabilization Kit (BD Biosciences). When indicated, naïve CD4 T cells were labeled prior to culture with 0.5 μM CellTrace Violet (Thermo Fischer Scientific). Flow cytometry was performed by using a LSRII Fortessa cytometer (BD Biosciences) or MACSQuant analyzer 10 (Myltenyi) and the data were analyzed by using FlowJo software (Tree Star).

### Mouse Phenotyping

To determine the frequency and phenotype of immune cells in primary and secondary lymphoid organs, thymus, spleen and lymph nodes were collected from Setdb1−/− and Setdb1+/+ mice and single-cell suspensions were obtained by mechanical disruption. Cells were then incubated on ice in FACS buffer (PBS, 3 mM EDTA, 3% fetal calf serum) containing 10 μg/mL anti-CD16/32 antibody for 20 minutes. Fluorochrome-conjugated antibodies were added at saturating concentrations, and cell suspensions were incubated on ice and protected from light for a further 20 minutes. For intracellular staining, cells were fixed and permeabilized by using the Transcription Factor Staining Buffer Set (Thermo Fischer Scientific) according to the manufacturer's instructions. The following antibodies were used for phenotyping: anti-TCR-β (H57-597), anti-CD4 (GK1.5), anti-NKp46 (29A1.4), anti-CD11b (M1/70), anti-CD19 (1D3), anti-CD25 (PC61), anti-CD69 (H1.2F3), anti-Siglec-F (E50-2440), anti-H-2Kb (AF6-88.5) and anti-Ki67 (B56) all from BD Biosciences; anti-CD8β (ebioH35-17.2), anti-CD8ϵ (53-6.7), anti-PDCA1 (ebio927), anti-I-A/I-E (M5/114), anti-CD11c (N418), anti-Gr1 (RB6-8C5), anti-B220 (RA3-6B2), anti-CD44 (IM7) and anti-CD62L (MEL-14), all from Thermo Fischer Scientific. Dendritic cells (DC) were gated based on I-Ab and CD11c expression (CD19-TCR-β-CD11c+I-Ab+) and CD8α, CD11b and PDCA-1 were used as markers to identify the conventional type 1 (cDC1, CD8α+CD11b−), conventional type 2 (cDC2, CD8α-CD11b+) and plasmacytoid (pDC, PDCA-1+) sub-populations. Monocytes/macrophages (Macro) and B cells (B cell) were defined as lin-CD11c-CD11b+SSC-AlowGr-1low/- and TCR-β-CD19+B220+, respectively. Neutrophils (Neutro), natural killer cells (NK) and eosinophils (Eosino) were identified as lin-CD11c-CD11b+SSC-AhighGr-1+, TCR-β-NKP46+ and lin-CD11c-CD11b+SSC-AhighGr-1-, respectively. Flow cytometry was performed by using a LSRII Fortessa cytometer (BD Biosciences) and the data were analyzed by using FlowJo software (Tree Star).

### Ex Vivo Measurement of Apoptosis

Single-cell suspensions of spleen, mesenteric lymph nodes and thymus were obtained as described above. Spleen and lymph node cells were labeled with antibodies specific for TCR-β (H57-597, Thermo Fischer Scientific) and CD4 (GK1.5, BD Biosciences), whereas thymocytes were stained with antibodies specific for CD4 and CD8β (ebioH35-17.2, ThermoFischer Scientific). Apoptotic cells were then labeled by using the Cell Event™ Caspase-3/7 Green Detection Reagent (Thermo Fischer Scientific) according to the manufacturer's instructions. Flow cytometry was performed by using a LSRII Fortessa cytometer (BD Biosciences) and the data were analyzed by using FlowJo software (Tree Star).

### Measurement of Cytokines in Cell Culture Supernatants

Following 6 days of culture in Th1 polarizing conditions, T cells were collected and extensively washed in complete medium. The differentiated cells (7.5×104 per well) were cultured overnight in 96-well flat bottom plates coated with anti-CDR antibody in complete culture medium containing anti-CD28 antibody. The concentrations of cytokines in the cell culture supernatants were then measured by flow cytometry using the FlowCytomix Kit (a bead-based multiple cytokine detection system) according to manufacturer's instructions (FlowCytomix, eBiosciences). Flow cytometry was performed by using a MACSquant Q10 flow cytometer (Miltenyi).

### Western Blotting

The different subpopulations of thymocytes were sorted on a FACS Aria (BD Biosciences) based on their expression of CD4 and CD8. Naïve CD4 T cells were purified from the spleen as described above. Cells were lysed in 1× NuPAGE LDS sample buffer and 1× NuPAGE sample reducing agent (Thermo Fischer Scientific). Whole cell lysates were then sonicated briefly and proteins were separated by SDS-PAGE on 4-12% Bis-Tris gels (Thermo Fischer Scientific), transferred onto nitrocellulose membranes (BA-S 83 Optitran, GE Healthcare Life Sciences), and probed with antibodies specific for SETDB1 (ab107225, Abcam), total H3 (ab1791, Abcam), H3K9me3 (D4W1U, Cell Signaling Technology) or beta ACTIN (ab8227, Abcam). The bands were detected by using Amersham ECL western blotting detection reagent (GE Healthcare Life Sciences) and the ChemiDoc XRS+ imaging system (Bio-Rad) after staining with secondary antibodies coupled to horseradish peroxidase. Images were analyzed by using Image Lab software (Bio-Rad).

### RNA-Seq Sample Preparation and Analysis

Total RNA was extracted by using the RNeasy Micro Kit (Qiagen) and its quality was assessed on a 2100 Bioanalyzer (Agilent Technologies). Only high-quality RNA (i.e. RNA of integrity number >7) was subsequently used to prepare the libraries according to the ScriptSeq RNA-seq protocol (Illumina). Quality controls of the libraries were performed by using standard methods, including quantification by Qubit (Thermo Fisher Scientific) and assessment of size distribution by using the 2100 Bioanalyzer. Samples were indexed and sequenced on an Illumina HiSeq 2500 or 3000 (paired-end reads of 100 or 150 bp, respectively). After trimming of adaptor sequences (Cutadapt 1.3) and removal of low-quality bases (−q value, <15), high-quality reads were aligned to the mouse reference genome mm10 (Genome Reference Consortium) by using TopHat version 2.0.5 (Trapnell et al., 2009). Count of the reads mapping to each gene was performed using Htseq-count. Differential expression analysis was performed by using the DESeq package (Bioconductor software) (Anders and Huber, 2010). An adjusted P value of <0.1 (P value adjusted for multiple testing with the Benjamini-Hochberg procedure) was used as cutoff to select the genes differentially expressed.

### ChIP, Semi-Quantitative PCR and Library Preparation and Sequencing

ChIP was performed as previously described (Lee et al., 2006). Briefly, following cell lysis, the chromatin was sonicated with a Bioruptor Pico (Diagenode) to obtain fragments of 100-300 bp. In each assay, we used 5-50 million cells and 2-10 μg of antibody specific for H3K9me3 (ab8898, Abcam) or H3K4me1 (ab8895, Abcam). Immunoprecipitation was performed by using Dynabead® Protein G (Thermo Fisher Scientific). Library preparation was carried out by using the TruSeq ChIP Sample Preparation Kit (Illumina). Library quality was assessed by using the 2100 Bioanalyzer and sequencing was performed on an Illumina HiSeq 3000 (paired-end reads of 150 bp). When indicated, semi-quantitative PCR was performed on a Light Cycler® 480 (Roche) using LightCycler 480 SYBR Green I Master (Roche). Primers specific for Ifng CNS17-20 (forward: tccctagactctgccactct; and reverse: gctcaccatcaataggcgtg) and for the glyceraldehyde-3-phosphate dehydrogenase (Gapdh) promoter (forward: gctccttgcccttccagatt and reverse: cccttcccaccctgttcatc) were used. The results were expressed as the percentage of input DNA normalized to the signal from the Gapdh promoter.

### ChIP-Seq Data Processing

Reads were filtered as described for RNA-seq and aligned to the mm10 reference genome by using BWA v.0.7.10 (Li and Durbin, 2009). H3K4me1 peaks were detected by using the ‘broad’ option of MACS2 v.2.1.0 (Zhang, 2008). To detect H3K9me3 peaks, we used the R Bioconductor package CSAW v.1.4.1 (Lun and Smyth, n.d.). The minimum mapping score was set to 50 and a window size of 300 was used. Differential binding windows were clustered in regions with the ‘mergeWindows’ function and the Benjamini-Hochberg method was applied to control the False Discovery Rate (FDR) across all detected clusters (‘combineTests’ function).

### Bioinformatics Analyses

R (https://www.R-project.org), SAMtools (Li et al., 2009) and the BEDtools suite v2.22.1 (Quinlan and Hall, 2010) were used to analyze high-throughput sequencing files. To determine the genome-wide distribution of H3K9me3 peaks, we defined the different genomic regions as follows: gene body coordinates were extracted from assembly GRCm38; promoters were defined as transcription start sites +1 kb/−2 kb; enhancers were identified as H3K4me1 peaks with no overlap with promoters; ERV coordinates were rebuilt from the RepeatMasker database as described below. As a control, we randomly distributed H3K9me3 peaks through the genome using the shuffle sub-command of the BEDtools suite. R package ‘Genomation’ was used to visualize genomic intervals (Akalin et al., 2015). Biological functions analysis of H3K9me3 ChIP-seq peak coordinates was performed by using the Genomic Regions Enrichment of Annotations Tool (GREAT) (McLean et al., 2010) with default settings and using the ‘single nearest gene’ option (each gene is assigned a regulatory domain that extends in both directions (within 100 kb) to the midpoint between the gene's TSS and the nearest gene's TSS but no more than the maximum extension in one direction). For Gene Ontology analysis the “enrichment analysis” tool from the Gene Ontology Consortium was used (http://geneontology.org). For analysis of motif enrichment, we used AME software of the MEME Suite version 4.11.3 with defaults options (0.05≥adjusted p-value) (McLeay and Bailey, 2010); the HOCOMOCOv10 MOUSE was used as the input transcription factor motif database. We also used the gene set enrichment analysis (GSEA) software (http://software.broadinstitute.org/gsea/index.jsp) with default settings except for the ‘Collapse dataset to gene symbols’ and ‘the permutation type’ which were set as ‘false’ and ‘gene set’, respectively. Heat maps were generated by using matrix2png version 1.2.1 (http://www.chibi.ubc.ca/matrix2png).

### ERV Reconstruction

Annotations of ERV elements were downloaded from the UCSC Genome Browser (assembly GRCm38, release of RepeatMasker: 2012 Feb. 7). We used the four major subfamilies (ERV1, ERVK, ERVL and ERVL-MaLR) of LTRs and excluded elements for which the curator was unsure of the classification. We merged ERV fragments from the same family (identical ‘repName’) into a single ERV when located within 20 bp, as previously described (Goke et al., 2015). Count of the reads mapping to each ERV was performed using Htseq-count (Anders et al., 2015) and normalization was performed with DESeq. ERVs with an expression score ≥1 were considered as expressed.

### Quantification and Statistical Analysis

Statistical parameters including the exact value and significance of n and precision measures (Mean+/−SEM or SD) as well as statistical significance are reported in the figures and figure legends. Unless stated otherwise, asterisks denote statistical significance as calculated by Student's t test in GraphPad PRISM 6 (*, p<0.05; **, p<0.01; ***, p<0.001). When large sets of unpaired data were compared, Pearson's chi-squared test was calculated in R to determine whether the observed difference between the sets of data arose by chance.

### Data Resources

Raw and processed data files from ChIP-seq and RNA-seq experiments have been deposited in the NCBI Gene Expression Omnibus (http://www.ncbi.nlm.nih.gov/geo/) under accession number GSE101546.

### Results

### Enhanced Th1 Priming in the Absence of SETDB1

To analyze the role of SETDB1 in

CD4 T cell differentiation and plasticity, we generated mice homozygous for a LoxP-flanked Setdb1 allele and expressing (Setdb1−/−), or not (Setdb1+/+), the CRE recombinase under the control of the Cd4 promoter. This strategy resulted in the almost complete absence of SETDB1 protein from CD4 single-positive (SP) thymocytes (data not shown). As SETDB1-deficiency was not compensated by overexpression of the other methyltransferases targeting H3K9 (data not shown), we also observed a marked loss of trimethylated H3K9 from naïve Setdb1−/− CD4 T cells (data not shown). The use of the Cd4-Cre transgene, which induces SETDB1 deletion relatively late in ontogeny, allowed for normal intrathymic T cell development. The total number of cells in the thymus, the relative proportions of the four main populations of thymocytes, and the proportion of semi-mature and mature CD4 SP cells were similar in SETDB1-deficient mice and control littermates (data not shown). In previous studies, by contrast, deletion of Setdb1 at the very early CD4/CD8 double-negative (DN) stage severely hampered T cell development in mice (Martin et al., 2015; Takikita et al., 2016).

In peripheral lymphoid tissues, we detected no consequences of T cell-specific SETDB1-deficiency on other populations of immune cells (data not shown). SETDB1 was previously implicated in the survival in various cell types (Collins et al., 2015; Karimi et al., 2014; Sarraf and Stancheva, 2004). For example, conditional deletion of the enzyme in mice expressing the CRE recombinase under the control of the Mb1 promoter abolished the B cell lineage (Collins et al., 2015). The impact of Setdb1 deletion on T cell survival was less pronounced: despite substantially increased activity of caspase-3/7, we only observed a partial loss of the T cell pool (data not shown), and the proportions of naïve and memory CD4 T cells were similar in Setdb1−/− and Setdb1+/+ mice (data not shown).

To obtain a more global view of the changes in gene expression induced by Setdb1 deletion, we performed directional RNA sequencing (RNA-seq) on FACS-sorted naïve Setdb1−/− and Setdb1+/+ CD4 T cells. Most of the differentially expressed genes were upregulated in Setdb1−/− cells (data not shown), consistent with a globally repressive effect of H3K9me3 on gene transcription. Among the upregulated genes, those involved in cell division were particularly enriched (data not shown). The proportion of CD4 T cells expressing the nuclear antigen Ki-67 (a marker of proliferating cells) being higher in Setdb1−/− mice than in control Setdb1+/+ littermates (data not shown), this upregulation of cell division-related genes most likely resulted from the observed lymphopenia rather than from a direct impact of Setdbl deletion on the regulation of these genes. Moreover, our ChIP-seq analysis found no particular enrichment for H3K9me3 peaks at cell cycle-related genes in naïve CD4 T cells (data not shown).

To assess if Setdb1 deletion could affect T cell function, we next analyzed the differential transcription of a gene set related to T helper cell activation and differentiation in our RNA-seq dataset. We found no major differences in the expression of these T helper-related genes between Setdb1−/− and Setdb1+/+ cells (data not shown), despite the presence of H3K9me3 peaks close to loci involved in lymphocyte-mediated immunity (data not shown). The vast majority of the genes were equally expressed in Setdb1−/− and Setdb1+/+ cells, most of the differentially expressed loci were transcribed at very low levels, and no lineage-specific transcriptomic signature appeared when focusing on deregulated genes. This lack of effect of SETDB1 deficiency on naïve CD4 T cell programming was further confirmed when we analyzed the production of lineage-specific mediators. Upon acute stimulation in neutral conditions, neither Setdb1−/− nor Setdb1+/+ cells produced Th1- or Th2-related cytokines spontaneously (data not shown). Together, these observations show that gene expression is very similar in Setdb1−/− and Setdb1+/+ naïve CD4 T cells, and that SETDB1-deficient cells are not a priori biased toward a specific T helper lineage.

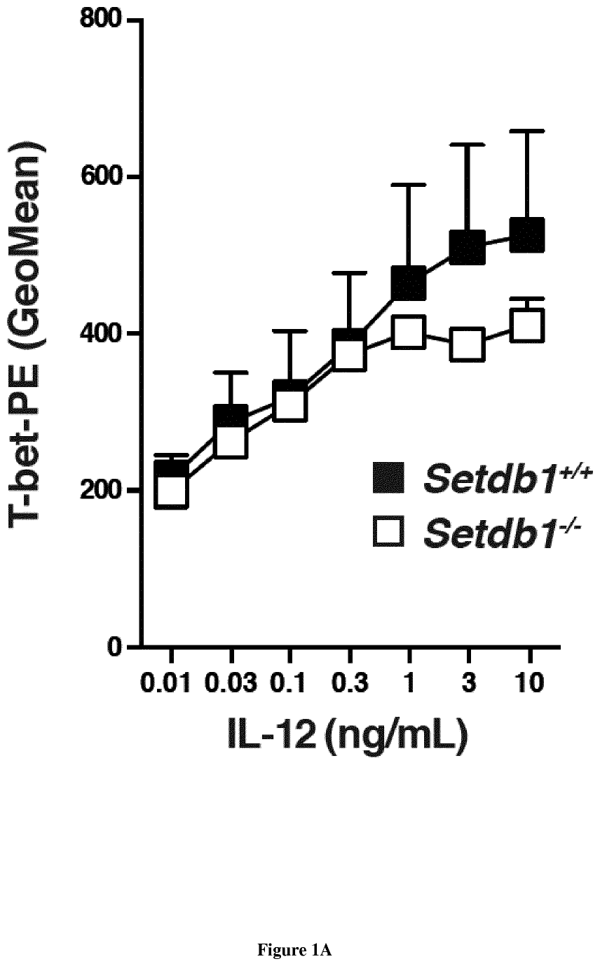

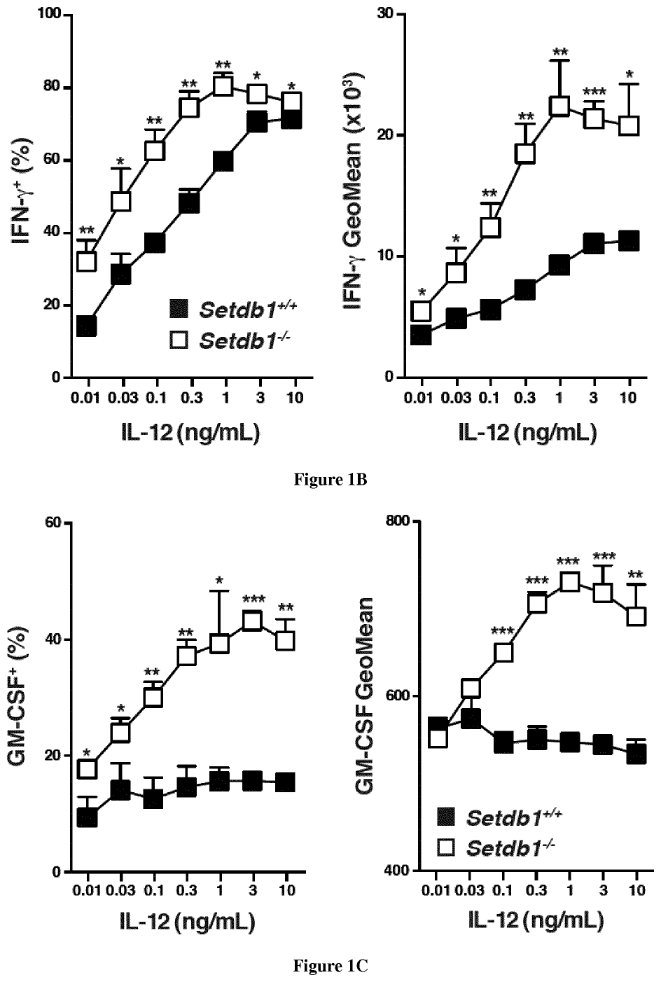

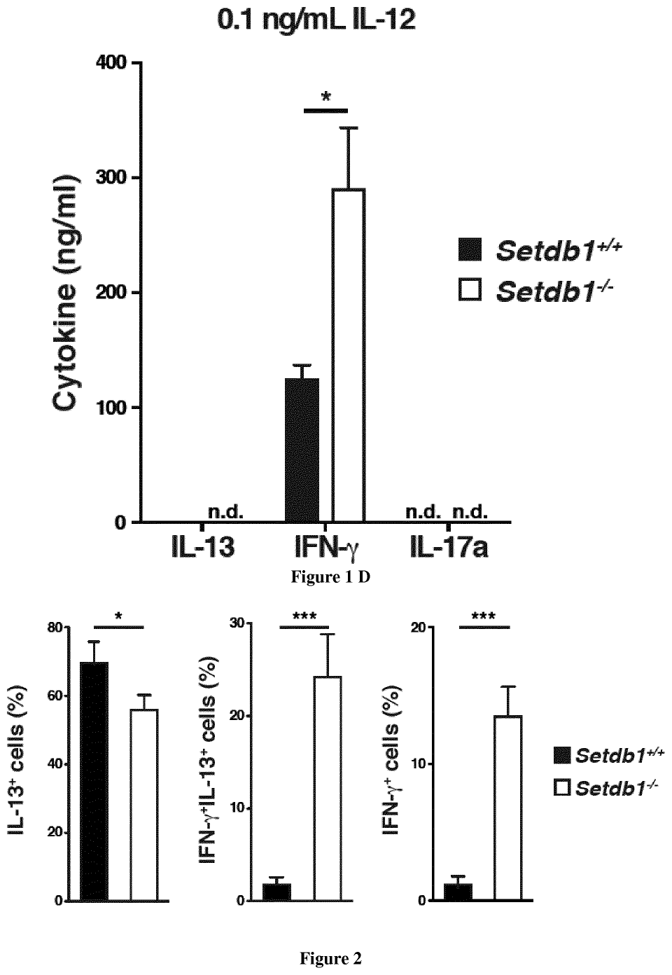

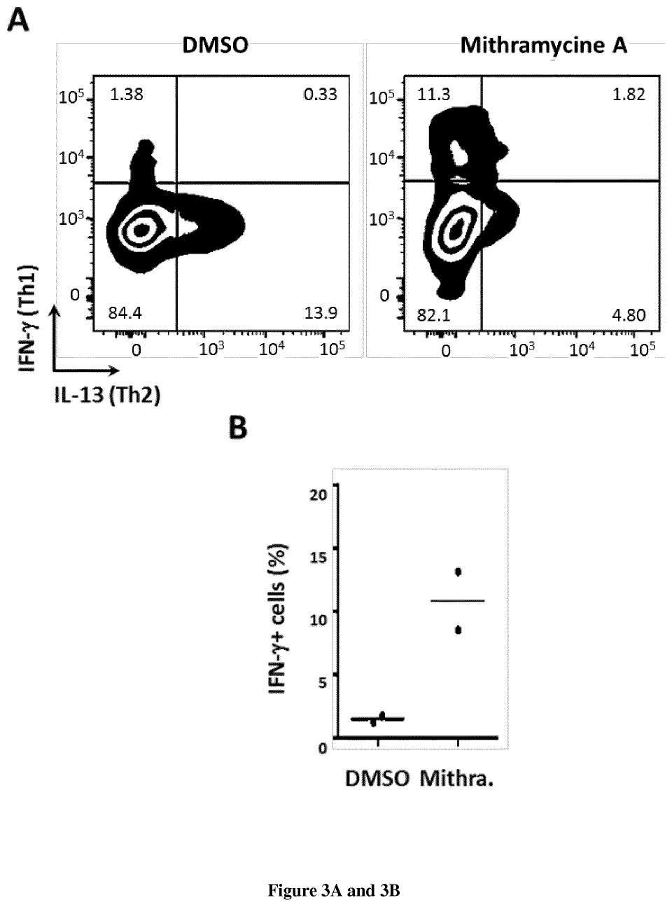

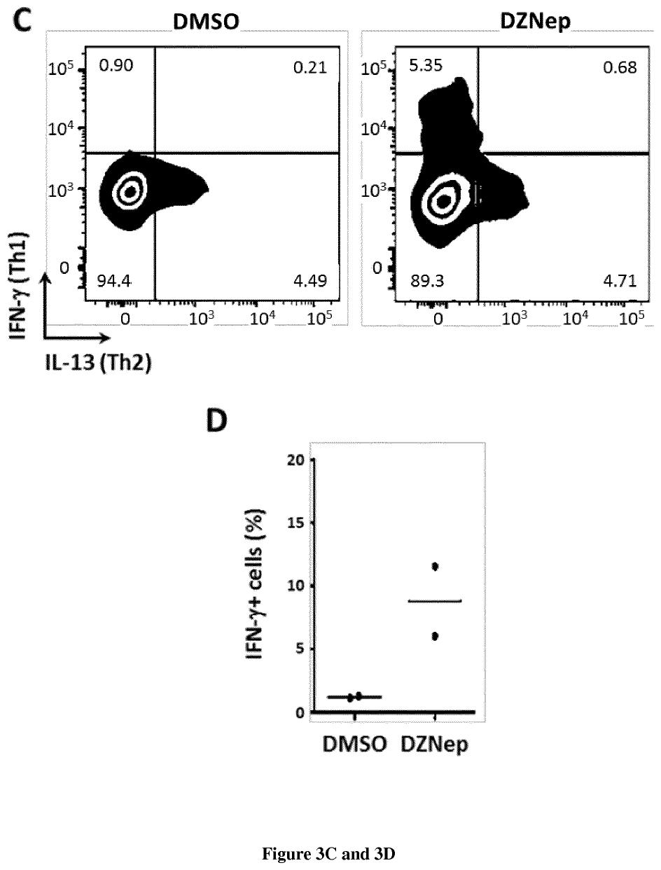

To test whether SETDB1 regulates cell lineage commitment in response to environmental signals, we analyzed Setdb1−/− and Setdb 1+/+ CD4 T cell fate in an IL-12-mediated Th1 differentiation assay. As expected from our experiments measuring caspase activity ex vivo (data not shown), SETDB1 deficiency impaired T cell survival at early time points (data not shown). However, a significant proportion of cells remained viable and showed normal activation upon TCR triggering, as measured by the upregulation of CD25 and CD69 (data not shown). As T cell differentiation depends on cell cycle progression, we analyzed the proliferative response of activated Setdb1−/− and Setdb1+/+ CD4 T cells by using a cell-tracing reagent, CellTrace Violet (CTV), to identify different generations of proliferating cells based on dye dilution. There was no difference between control and mutant cells (data not shown), which had similar proliferation indexes and percentages of divided cells (data not shown). Upon exposure to increasing doses of IL-12, Setdb1−/− cells also expressed T-bet, the Th1 master regulator, at a similar level as their Setdb1+/+ counterparts (FIG. 1A). Together, these observations indicate that SETDB1 deficiency does not affect naïve CD4 T cell activation, proliferation and commitment to the Th1 lineage. It does however lead to greater acquisition of lineage-specific functions. At all tested concentrations of IL-12, the percentage of cells producing Th1-related cytokines and the amount of cytokine synthesized per cell were higher in Setdb1−/− than in Setdb1+/+ cells (FIGS. 1B, C and D). This exacerbated production of cytokines was not the result of a global transcriptional derepression as we saw no aberrant secretion of soluble mediators related to alternative lineages (FIG. 1D). Together these results highlight a key role for SETDB1 in regulating the magnitude of the Th1 response.

### Normal Acquisition of the Th2 Phenotype by SETDB1-Deficient Cells

Most of the genes encoding lineage-specific cytokines in naïve CD4 T cells have both permissive and repressive epigenetic marks on their promoters and enhancers. They are thus poised for transcription to guarantee the pluripotency while also preserving the identity of the cells. The enhanced Th1 response observed in Setdb1−/− cells might, therefore, result from a loss of H3K9me3 at these cis-regulatory regions, and this may potentially affect other lineages. To test this hypothesis, we cultured Setdb1−/− and Setdb1+/+ naïve CD4 T cells in Th2 polarizing conditions, with exogenous IL-4 as lineage-instructive signal. The proliferative response and viability at day six of Setdb1−/− and Setdb1+/+ cells were similar (data not shown). Moreover, Setdb1−/− cells committed to the Th2 lineage as completely as did their control counterparts, with almost all cells expressing the master regulator of the Th2 lineage GATA-3 (data not shown) and no aberrant expression of the Th1-specifying transcription factor T-bet (data not shown). Production of IL-13, a Th2-specific cytokine, was also similar in Setdb1−/− and Setdb1+/+ cells (data not shown). Thus, in contrast to what we observed in Th1 polarizing conditions, there was no enhanced production of Th2 lineage-specific mediators by SETDB1-deficient cells grown in the presence of IL-4. Therefore, this lysine methyltransferase seems not to play a key role in establishing the Th2-specific gene expression program.

### SETDB1 is Required for Stable Th2 Cell Commitment

Interestingly, unlike their wild-type counterparts, Setdb1−/− cells grown in Th2-polarizing conditions produced small amounts of the Th1 cytokine IFN-γ (data not shown). This observation correlated with lower expression of the Th2-promoting transcription factor GATA-3 in the Setdb1−/− cells than in the Setdb1+/+ cells (data not shown), suggesting that SETDB1 may have a role in naïve CD4 T cell commitment to the Th2 phenotype. This IFN-γ ‘leak’ may result from defective repression of Th1-related loci in Th2 cells, which could potentially lead to functional and phenotypic instability. To assess if SETDB1 might control Th2 cell plasticity, we cultured Setdb1−/− and Setdb1+/+ cells for six days in Th2-polarizing conditions, washed them extensively, and then cultured them in Th1-polarizing conditions. In agreement with the Th1/Th2 paradigm, the control Setdb1+/+ Th2 cells remained phenotypically and functionally stable after two days of culture in Th1 conditions; they still produced large amounts of the Th2-type cytokine IL-13 and had not switched on IFN-γ production (FIG. 2A). By contrast, a large fraction of the Setdb1−/− cell population secreted IFN-γ; this phenomenon was even more pronounced after four days of culture (FIG. 2A). IFN-γ secretion was accompanied by downregulation of GATA-3 and upregulation of T-bet (data not shown). In fact, SETDB1-deficiency allowed the virtually complete reprogramming of Th2 cells upon exposure to Th1-instructing signals, with extinction of Th2 gene expression and induction of the entire Th1 gene set (data not shown).

SETDB1 plays a key role in silencing ERVs (Bulut-Karslioglu et al., 2014; Matsui et al., 2010). Ectopic expression of these retrotransposons can lead to activation of nucleic acid-sensing by the innate immune system and, eventually, to production of the type I IFNs such as IFN-γ and IFN-γ (Chiappinelli et al., 2015). Together with IL-12 and IFN-γ, type I IFNs can reprogram Th2 cells into stable cells producing IFN-γ and expressing both GATA-3 and T-bet (Hegazy et al., 2010). Activation of ERVs in SETDB1-deficient Th2 cells might thus account for the trans-differentiation into the Th1 lineage that we observed: ERV-induced secretion of IFN-γ and IFN-γ in combination with exogenous IL-12 and the observed aberrant production of IFN-γ might reprogram the Th2 cells. We found no aberrant levels of type I IFN mRNA in SETDB1-deficient cells, however (data not shown), and neutralization of IFN-γ did not prevent Setdb1−/− Th2 cells from switching to a Th1 phenotype (data not shown).

To assess more directly if ectopic expression of Th1-instructive mediators by Setdb1−/− Th2 cells might account for their phenotypic instability, we co-cultured Setdb1−/− and Setdb1+/+ Th2 cells in Th1-polarizing conditions. In this setting, the SETDB1-deficient cells still showed substantial plasticity while their control counterparts did not (data not shown). Although the IL-12 added to the culture and the pro-Th1 mediators secreted by Setdb1−/− cells partly antagonized the Th2 program, they failed to switch on the Th1 gene network in Setdb1+/+ cells. Together, these data clearly identify SETDB1 as a key controller of Th2 cell commitment through a cell-intrinsic mechanism that is not limited to direct or indirect type I and/or type II IFN genes silencing.

### SETDB1-Dependent H3K9 Trimethylation at a Subset of ERVs

To determine how SETDB1 controls Th2 cell commitment and stability, we first performed high-throughput chromatin immunoprecipitation (ChIP) for genome-wide mapping of H3K9me3 in Setdb1+/+ Th2 cells. We observed no more peaks at genes bodies or promoters than would be expected by a random distribution (data not shown). By contrast, we found substantial and statistically significant enrichment of H3K9me3 peaks at enhancers (defined as non-promoter H3K4me1+genomic regions) and at ERVs (data not shown). Unlike what was observed previously in mouse embryonic stem cells (Mikkelsen et al., 2007), in Th2 cells the repertoire of ERVs marked by H3K9me3 was not specifically enriched for ERV1 and ERVK: the representation of the three classes of retroviruses in the mouse genome was similar to that of the 17,556 retrotransposons (1.9% of total) associated with H3K9me3 (data not shown). Interestingly, 73% of the peaks at enhancers also overlapped with ERVs (data not shown), suggesting that the ERVs rather than the enhancers themselves may be the targets for H3K9 trimethylation. To test this hypothesis, we analyzed the distribution of H3K9me3 across the length of individual ERV and enhancer sequences. The H3K9me3 signal clearly peaked at and aligned with the center of the ERVs (data not shown). By contrast, the signal appeared randomly distributed across the enhancer sequences with only a little accumulation on the flanking regions of these cis-regulatory elements (data not shown). We next calculated the distance between the center of these genomic regions and the center of the H3K9me3 peaks in cases where the ERVs and enhancers overlapped and where the two genomic regions were mutually exclusive (data not shown). In both cases, the H3K9me3 peaks were closer to the ERVs than to the enhancers (data not shown). Moreover, we also observed significant H3K9me3 signal enrichment at enhancers that did not overlap with a peak of H3K9me3 but that were located close to an ERV marked by the repressive mark (data not shown). These data indicate that H3K9 trimethylation is directed at ERVs and only marks enhancers when these regions overlap or flank the retrotransposons.

To determine which lysine methyltransferase is necessary for H3K9me3 deposition at ERVs in Th2 cells, we compared H3K9me3 deposition at ERVs in SETDB1-deficient Th2 cells with that in SUV39H1-deficient Th2 cells by ChIP sequencing. Whereas SUV39H1-deficiency had no significant impact on H3K9me3 deposition at ERVs in Th2 cells, most, if not all of the peaks vanished in the SETDB1-deficient cells (data not shown). Together, these data show that SETDB1 targets H3K9me3 at a subset of ERVs in Th2 cells, and that some of these retrotransposons overlap or flank enhancers.

### SETDB1-Deficiency Upregulates ERVs and Their Neighboring Genes

Recent studies have strengthened the hypothesis that transposable elements have been co-opted for the regulation of host gene networks (Chuong et al., 2017; 2016). The impact of SETDB1 deletion on CD4 T cell fate might, therefore, result from a loss of H3K9me3 at ERVs that behave as cis-regulatory modules of transcription, and/or regulate the activity of enhancers.

To test this hypothesis, we analyzed the consequences of SETDB1 deletion on ERV activity, on the status of their nearest enhancers, and on the expression of associated genes. We first compared ERV expression levels in Setdb1−/− and Setdb1+/+ Th2 cells. To avoid any bias, we excluded from the analysis transposable elements located on gene bodies and those that overlapped with promoters. In Setdb1−/− Th2 cells, the expression of 22% of the ERVs that lost H3K9me3 was deregulated, 77% of which were overexpressed at low levels in Setdb1−/− cells (data not shown). As we observed that H3K9me3 signal spread from ERV to close enhancers (data not shown), we hypothesized that the cis-regulatory elements could also be de-repressed in Setdb1−/− cells. We thus analyzed bidirectional transcription of enhancers located in the vicinity of ERVs marked by H3K9me3 in Setdb1+/+ cells and upregulated in Setdb1−/− cells. The enhancers overlapping or flanked by ERVs that were activated (i.e. transcribed) following H3K9me3 disappearance were themselves more expressed in Setdb1−/− than in Setdb1+/+ Th2 cells (data not shown). Together these data show that SETDB1 deletion leads to loss of H3K9me3 from ERVs, and to the concomitant activation of these retroelements and their neighboring enhancers. Finally, we tested if this cascade of events resulted in deregulation of gene expression. Our analysis clearly demonstrated that the genes associated with enhancers flanking or overlapping ERVs that showed overexpression in SETDB1-deficient cells were also significantly upregulated (data not shown). We conclude that SETDB1-dependent H3K9me3 deposition at ERVs inactivates neighboring enhancers and concomitantly participate to the silencing of their target genes.

### SETDB1-Dependent H3K9 Trimethylation at ERVs Represses Th1-Specific Genes

In our functional assays, SETDB1 deletion led to enhanced Th1 priming and to Th2 cell instability (FIGS. 1A-D and 2). As discussed above, loss of regulation of the Th1 gene network could explain these observations. Based on our epigenetic and transcriptomic studies, we postulated that SETDB1 might control Th1 gene expression by repressing ERVs acting as cis-regulatory elements of these genes and/or regulating the activity of their enhancers. To test this hypothesis, we first used GREAT (Genomic Regions Enrichment of Annotations Tool) (McLean et al., 2010) to analyse the set of ERVs marked by H3K9me3 in a SETDB1-dependent manner. GREAT assigns biological significance to a set of non-coding genomic regions by analyzing the annotations of their nearby genes. To avoid any bias, we excluded the transposable elements overlapping Th2 gene enhancers. We observed a strong association of the retrotransposons with genes involved in immune processes, including leukocyte activation and cytokine production (data not shown). Interestingly, this distribution was cell-type specific: there was very little overlap between the ERVs marked by H3K9me3 in Th2 cells and those in adipocytes (data not shown), and the ERVs marked by H3K9me3 in white adipose cells were associated with genes that have no direct link with immunity (data not shown). In Th2 cells, thus, SETDB1 is responsible for H3K9 trimethylation at a restricted and cell type-specific set of ERVs that are associated with genes that control T cell functions. Motif enrichment analysis of H3K9me3-marked ERV sequences strengthened this observation; it revealed a strong enrichment for the binding sites of Th1-related transcription factors, including STAT1, FOXO3, IRF1 and STAT4 (data not shown). The two STAT proteins are mobilized upon exposure of cells to IFN-γ and IL-12. Crucially, they control naïve CD4 T cell differentiation into T helper cells by shaping the lineage-specific active enhancer repertoire (Vahedi et al., 2012). The unbiased “Ingenuity Upstream Regulator Analysis” of our RNA-seq data performed in IPA also identified these four transcription factors as very likely to be responsible for the differences in gene expression observed in Setdb1−/− vs Setdb1+/+ Th2 cells upon culture in Th1-polarizing conditions (data not shown). From these data, we conclude that the differences in stability observed between Setdb1−/− and Setdb1+/+ Th2 cells are very likely explained by SETDB1 causing H3K9me3 deposition at, and thus suppression of, ERVs that are located in the vicinity of genes involved in leukocyte function and that are targeted by Th1-specific transcription factors.

To test more directly if SETDB1-dependent H3K9me3 deposition at ERVs plays a role in the repression of the Th1 program and, as a consequence, in Th2 cell stability, we analyzed the location of the retroelements relative to lineage-specific enhancers, defined as non-promoter genomic regions showing significant enrichment for H3K4me1 in Th1 cells (Vahedi et al., 2012). We found 4397 putative Th1 enhancers associated with H3K9me3+ERVs in Th2 cells (data not shown). Interestingly, they included the previously identified conserved, non-coding sequences located 17-1-20 kb downstream of the Ifng gene (CNS17-20). While they are poised in naïve T cells, with a strong H3K4me1 signal flanked by a large domain of H3K9me3, they lose competence upon Th2 cell commitment, with an accumulation of H3K9me3 and a complete loss of H3K4me1 (data not shown). In SETDB1−/− cells, this enhancer as well as the thousands of others marked or flanked by an ERV, loose H3K9me3 and thus probably become accessible to the Th1-specific transcription factors mobilized downstream of the IFN-γ and IL-12 receptor. Consistent with this hypothesis, we found that the H3K4me1 signal at Ifng CNS17-20 was substantially higher in Setdb1−/− than in Setdb1+/+ Th2 cells (data not shown). It therefore appears that, in the absence of SETDB1, the lack of deposition of H3K9me3 at ERVs overlapping or flanking Th1-related enhancers makes them accessible to transcription factors, including IRF1, FOXO3 and lineage-associated STATs. This lack of repression potentially affects a large number of Th1-associated loci including those encoding IFN-γ and T-bet, the signature cytokine and so-called master regulator of the lineage, respectively. They also include those encoding other critical transcriptional regulators such as STAT4, IRF1, HLX and RUNX3 (data not shown). All of these genes have at least one enhancer associated with an ERV marked by H3K9me3 in Th2 cells and are, in addition, upregulated in SETDB1-deficient Th2 cells upon culture in Th1-inducing conditions. In conclusion, our data reveal for the first time that Th2 cell lineage stability is controlled at the level of the chromatin by the SETDB1-dependent deposition of H3K9me3 at a restricted set of ERVs flanking or behaving as Th1-gene enhancers.

### SETDB1 Inhibitors Alter Th2 Cell Commitment

To test whether the acute inhibition of SETDB1 could impact on Th2 cell differentiation and commitment, we next targeted SETDB1 in differentiating wild-type Th2 cells using non-specific inhibitors (DZNEP and Mithramycin). The use of these pharmacological agents results in the destabilization of the Th2 cells which start to produce pro-Th1 factor, in particular IFN-γ (FIGS. 3A, 3B, 3C and 3D). Interestingly, after 6 days of culture in the presence of mithramycine A, and without exposing the differentiating Th2 cells to a pro-Th1 signal, the Th2 cells start to produce IFN-γ and to decrease their production of IL-13 (pro-Th2 factor) (FIGS. 3A and 3B).

### CONCLUSION

In conclusion, we have shown here that the lysine methyltransferase SETDB1 controls CD4 T cell identity by repressing ERVs that flank or overlap Th1 lineage-specific enhancers. This enzyme is thus a potential target for drugs that might be useful, for example, to promote Th1 cell differentiation in various infectious diseases, or to prevent harmful Th2 responses in allergic disorders.

### REFERENCES

Throughout this application, various references describe the state of the art to which this invention pertains. The disclosures of these references are hereby incorporated by reference into the present disclosure.

Akalin, A., Franke, V., Vlahoviček, K., Mason, C. E., Schiibeler, D. (2015). Genomation: a toolkit to summarize, annotate and visualize genomic intervals. Bioinformatics 31, 1127-1129.

Allan, R. S., Zueva, E., Cammas, F., Schreiber, H. A., Masson, V., Belz, G. T., Roche, D., Maison, C., Quivy, J.-P., Almouzni, G., et al. (2012). An epigenetic silencing pathway controlling T helper 2 cell lineage commitment. Nature 487, 249-253.

Anders, S., Huber, W. (2010). Differential expression analysis for sequence count data. Genome Biol. 11, R106.

Anders, S., Pyl P. T., Huber W. (2015). HTSeq-a Python framework to work with high-throughput sequencing data. Bioinformatics 31, 166-169.

Becker, J. S., Nicetto, D., Zaret, K. S. (2016). H3K9me3-Dependent Heterochromatin: Barrier to Cell Fate Changes. Trends Genet. 32, 29-41.

Bilodeau, S., Kagey, M. H., Frampton, G.M., Rahl, P. B., Young, R. A. (2009). SetDB1 contributes to repression of genes encoding developmental regulators and maintenance of ES cell state. Genes Dev. 23, 2484-2489.

Bulut-Karslioglu, A., La Rosa-Velazquez, De, I. A., Ramirez, F., Barenboim, M., Onishi-Seebacher, M., Arand, J., Galan, C., Winter, G. E., Engist, B., Gerle, B., et al. (2014). Suv39h-dependent H3K9me3 marks intact retrotransposons and silences LINE elements in mouse embryonic stem cells. Mol. Cell 55, 277-290.

Chiappinelli, K. B., Strissel, P. L., Desrichard, A., Li, H., Henke, C., Akman, B., Hein, A., Rote, N. S., Cope, L. M., Snyder, A., et al. (2015). Inhibiting DNA Methylation Causes an Interferon Response in Cancer via dsRNA Including Endogenous Retroviruses. Cell 162, 974-986.

Chuong, E. B., Elde, N. C., Feschotte, C. (2017). Regulatory activities of transposable elements: from conflicts to benefits. Nat. Rev. Genet. 18, 71-86.

Chuong, E. B., Elde, N. C., Feschotte, C. (2016). Regulatory evolution of innate immunity through co-option of endogenous retroviruses. Science 351, 1083-1087.

Collins, P. L., Kyle, K. E., Egawa, T., Shinkai, Y., Oltz, E. M. (2015). The histone methyltransferase SETDB1 represses endogenous and exogenous retroviruses in B lymphocytes. Proc. Natl. Acad. Sci. USA. 112, 8367-8372.

García-Cao, M., O'Sullivan, R., Peters, A. H. F. M., Jenuwein, T., Blasco, M. A. (2003). Epigenetic regulation of telomere length in mammalian cells by the Suv39h1 and Suv39h2 histone methyltransferases. Nat. Genet. 36, 94-99.

Göke, J., Lu, X., Chan, Y.-S., Ng, H.-H., Ly, L.-H., Sachs, F., Szczerbinska, I. (2015). Dynamic Transcription of Distinct Classes of Endogenous Retroviral Elements Marks Specific Populations of Early Human Embryonic Cells. Cell Stem Cell. 16, 135-141.

Hegazy, A. N., Peine, M., Helmstetter, C., Panse, I., Fröhlich, A., Bergthaler, A., Flatz, L., Pinschewer, D. D., Radbruch, A., Lohning, M. (2010). Interferons Direct Th2 Cell Reprogramming to Generate a Stable GATA-3+T-bet+ Cell Subset with Combined Th2 and Th1 Cell Functions. Immunity 32, 116-128.

Imbeault, M., Helleboid, P.-Y., Trono, D. (2017). KRAB zinc-finger proteins contribute to the evolution of gene regulatory networks. Nature 543, 550-554.

Kanno, Y., Vahedi, G., Hirahara, K., Singleton, K., O'Shea, J. J. (2012). Transcriptional and Epigenetic Control of T Helper Cell Specification: Molecular Mechanisms Underlying Commitment and Plasticity. Annu. Rev. Immunol. 30, 707-731.

Karanth Archana Venkataramana, Radhika Radha Maniswami, Seema Prashanth, Hemalatha Govindaraj, Ramya Padmavathy, Sooriya Kumar Jegatheesan, Ramesh Mullangi and Sriram Rajagopal. Emerging role of SETDB1 as a therapeutic target. Expert opinion on therapeutic targets, 2017.

Karimi, M. M., Goyal, P., Maksakova, I. A., Bilenky, M., Leung, D., Tang, J. X., Shinkai, Y., Mager, D. L., Jones, S., Hirst, M., et al. (2014). DNA Methylation and SETDB1/H3K9me3 Regulate Predominantly Distinct Sets of Genes, Retroelements, and Chimeric Transcripts in mESCs. Cell Death Dis. 5, 676-687.

Lachner, M., O'Carroll, D., Rea, S., Mechtler, K., Jenuwein, T. (2001). Methylation of histone H3 lysine 9 creates a binding site for HP1 proteins. Nature 410, 116-120.

Lee, T. I., Johnstone, S. E., Young, R. A. (2006). Chromatin immunoprecipitation and microarray-based analysis of protein location. Nat. Protoc. 1, 729-748.

Li, H., Durbin, R. (2009). Fast and accurate short read alignment with Burrows-Wheeler transform. Bioinformatics 25, 1754-1760.

Li, H., Handsaker, B., Wysoker, A., Fennell, T., Ruan, J., Homer, N., Marth, G., Abecasis, G., Durbin, R., 1000 Genome Project Data Processing Subgroup (2009). The Sequence Alignment/Map format and SAMtools. Bioinformatics 25, 2078-2079.

Liu, J., Magri, L., Zhang, F., Marsh, N. O., Albrecht, S., Huynh, J. L., Kaur, J., Kuhlmann, T., Zhang, W., Slesinger, P. A., et al. (2015). Chromatin Landscape Defined by Repressive Histone Methylation during Oligodendrocyte Differentiation. J. Neurosci. 35, 352-365.

Loyola, A., Bonaldi, T., Roche, D., Imhof, A., Almouzni, G. (2006). PTMs on H3 variants before chromatin assembly potentiate their final epigenetic state. Mol. Cell 24, 309-316.

Loyola, A., Tagami, H., Bonaldi, T., Roche, D., Quivy, J.-P., Imhof, A., Nakatani, Y., Dent, S. Y. R., Almouzni, G. (2009). The HP1α-CAF1-SetDB1-containing complex provides H3K9me1 for Suv39-mediated K9me3 in pericentric heterochromatin. EMBO Reports 10, 769-775.

Lun, A. T. L., Smyth, G. K. (2016). csaw: a Bioconductor package for differential binding analysis of ChIP-seq data using sliding windows. Nucleic Acids Res. 44, e45-e45.

Martin, F. J., Xu, Y., Lohmann, F., Ciccone, D .N., Nicholson, T. B., Loureiro, J. J., Chen, T., Huang, Q. (2015). KMT1E-mediated chromatin modifications at the FcyRIIb promoter regulate thymocyte development. Genes Immun. 16, 162-169.

Matsui, T., Leung, D., Miyashita, H., Maksakova, I. A., Miyachi, H., Kimura, H., Tachibana, M., Lorincz, M. C., Shinkai, Y. (2010). Proviral silencing in embryonic stem cells requires the histone methyltransferase ESET. Nature 464, 927-931.

McLean, C. Y., Bristor, D., Hiller, M., Clarke, S. L., Schaar, B. T., Lowe, C. B., Wenger, A. M., Bejerano, G. (2010). GREAT improves functional interpretation of cis-regulatory regions. Nat. Biotechnol. 28, 495-501.

McLeay, R. C., Bailey, T. L. (2010). Motif Enrichment Analysis: a unified framework and an evaluation on ChIP data. BMC Bioinformatics 11, 165.

Mikkelsen, T. S., Ku, M., Jaffe, D. B., Issac, B., Lieberman, E., Giannoukos, G., Alvarez, P., Brockman, W., Kim, T.-K., Koche, R. P., et al. (2007). Genome-wide maps of chromatin state in pluripotent and lineage-committed cells. Nature 448, 553-560.

Mozzetta, C., Boyarchuk, E., Pontis, J., Ait-Si-Ali, S. (2015). Sound of silence: the properties and functions of repressive Lys methyltransferases. Nat. Rev. Mol. Cell Biol. 16, 499-513.

O'Shea, J. J., Lahesmaa, R., Vahedi, G., Laurence, A., Kanno, Y. (2011). Genomic views of STAT function in CD4+ T helper cell differentiation. Nat. Rev. Immunol. 11, 239-250.

O'Shea, J. J., Paul, W. E. (2010). Mechanisms Underlying Lineage Commitment and Plasticity of Helper CD4+ T Cells. Science 327, 1098-1102.

Peters, A. H., O'Carroll, D., Scherthan, H., Mechtler, K., Sauer, S., Schofer, C., Weipoltshammer, K., Pagani, M., Lachner, M., Kohlmaier, A., et al. (2001). Loss of the Suv39h histone methyltransferases impairs mammalian heterochromatin and genome stability. Cell 107, 323-337.

Peters, A. H. F. M., Mermoud, J. E., O'Carroll, D., Pagani, M., Schweizer, D., Brockdorff, N., Jenuwein, T. (2002). Histone H3 lysine 9 methylation is an epigenetic imprint of facultative heterochromatin. Nat. Genet. 30, 77-80.

Quinlan, A. R., Hall, I. M. (2010). BEDTools: a flexible suite of utilities for comparing genomic features. Bioinformatics 26, 841-842.

Rebollo, R., Karimi, M. M., Bilenky, M., Gagnier, L., Miceli-Royer, K., Zhang, Y., Goyal, P., Keane, T. M., Jones, S., Hirst, M., et al. (2011). Retrotransposon-Induced Heterochromatin Spreading in the Mouse Revealed by Insertional Polymorphisms. PLoS Genet. 7, e1002301.

Rowe, H. M., Kapopoulou, A., Corsinotti, A., Fasching, L., Macfarlan, T. S., Tarabay, Y., Viville, S., Jakobsson, J., Pfaff, S. L., Trono, D. (2013). TRIM28 repression of retrotransposon-based enhancers is necessary to preserve transcriptional dynamics in embryonic stem cells. Genome Res. 23, 452-461.

Saint-André, V., Batsché, E., Rachez, C., Muchardt, C. (2011). Histone H3 lysine 9 trimethylation and HP1γ favor inclusion of alternative exons. Nat. Struct. Mol. Biol. 18, 337-344.

Sarraf, S. A., Stancheva, I. (2004). Methyl-CpG binding protein MBD1 couples histone H3 methylation at lysine 9 by SETDB1 to DNA replication and chromatin assembly. Molecular Cell 15, 595-605.

Schultz, D. C., Ayyanathan, K., Negorev, D., Maul, G. G., Rauscher, F. J., III. (2002). SETDB1: a novel KAP-1-associated histone H3, lysine 9-specific methyltransferase that contributes to HP1-mediated silencing of euchromatic genes by KRAB zinc-finger proteins. Genes Dev. 16, 919-932.

Takikita, S., Muro, R., Takai, T., Otsubo, T., Kawamura, Y. I., Dohi, T., Oda, H., Kitajima, M., Oshima, K., Hattori, M., et al. (2016). A Histone Methyltransferase ESET Is Critical for T Cell Development. J. Immunol. 197, 2269-2279.

Trapnell, C., Pachter, L., Salzberg, S. L. (2009). TopHat: discovering splice junctions with RNA-Seq. Bioinformatics 25, 1105-1111.

Tumes, D. J., Onodera, A., Suzuki, A., Shinoda, K., Endo, Y., Iwamura, C., Hosokawa, H., Koseki, H., Tokoyoda, K., Suzuki, Y., et al. (2013). The Polycomb Protein Ezh2 Regulates Differentiation and Plasticity of CD4+ T Helper Type 1 and Type 2 Cells. Immunity 39,819-832.

Vahedi, G., Takahashi, H., Nakayamada, S., Sun, H.-W., Sartorelli, V., Kanno, Y., O'Shea, J. J. (2012). STATs shape the active enhancer landscape of T cell populations. Cell 151, 981-993.

Vakoc, C. R., Mandat, S. A., Olenchock, B. A., Blobel, G. A. (2005). Histone H3 Lysine 9 Methylation and HP1γ Are Associated with Transcription Elongation through Mammalian Chromatin. Mol. Cell 19, 381-391.

Wilson, C. B., Rowell, E., Sekimata, M. (2009). Epigenetic control of T-helper-cell differentiation. Nat. Rev. Immunol. 9, 91-105.

Xiao, X., Shi, X., Fan, Y., Wu, C., Zhang, X., Minze, L., Liu, W., Ghobrial, R. M., Lan, P., Li, X. C. (2016). The Costimulatory Receptor OX40 Inhibits Interleukin-17 Expression through Activation of Repressive Chromatin Remodeling Pathways. Immunity 44, 1271-1283.

Young, H. A., Ghosh, P., Ye, J., Lederer, J., Lichtman, A., Gerard, J. R., Penix, L., Wilson, C. B., Melvin, A. J., McGurn, M. E. (1994). Differentiation of the T helper phenotypes by analysis of the methylation state of the IFN-gamma gene. J Immunol. 153, 3603-3610.

Zhang, Y., Tao, L., Meyer, C.A ., Eeckhoute, J., Johnson, D. S., Bernstein, B. E., Nusbaum, C., Myers, R. M., Brown, M., Li, W., et al. (2008). Model-based Analysis of ChIP-Seq (MACS). Genome Biol. 9, R137.

Zhu, Y., van Essen, D., Saccani, S. (2012). Cell-type-specific control of enhancer activity by H3K9 trimethylation. Mol. Cell 46, 408-423.

1. (canceled)

2. The method according to claim 11, wherein the step of administering results in an increase in a Th1 response of an immune response.

3. (canceled)

4. The method according to claim 5, wherein the T-helper type 2 (Th2)-mediated disease is cancer or an infectious disease.

5. A method of treating a T-helper type 2 (Th2)-mediated disease in a subject in need thereof, comprising
administering to the subject a therapeutically effective amount of a SETDB1 inhibitor.

6. The method according to claim 5 wherein the T-helper type 2 (Th2)-mediated disease is an allergic disorder or asthma.

7. The method according to claim 5, wherein the SETDB1 inhibitor is administered in combination with an immune adjuvant inducting and/or promoting Th1 cell differentiation.

8. The method according to claim 7, wherein the immune adjuvant is IL-12, LPS, Complete Freund's adjuvant, or an Aluminium salt.

9. The method according to claim 5, wherein the SETDB1 inhibitor is selected from the group consisting of mithramycin, mithramycin analogs, 3-Deazaneplanocin A and paclitaxel.

10. (canceled)

11. A method of increasing the Th1/Th2 ratio of an immune response in a subject in need thereof, comprising
administering to the subject a therapeutically effective amount of a SETDB1 inhibitor, wherein the therapeutically effective amount is sufficient to increase the Th1/Th2 ratio of the immune response of the subject.

12. The method according to claim 6 wherein the asthma is allergic asthma.

13. The method of claim 8, wherein the aluminium salt is Alum, CpG or squalene.
